(19) United States
(12) Patent Application Publication
(10) Pub. No.: US 2016/0030223 A1
(43) Pub. Date: Feb. 4, 2016

(54) ANKLE FOOT ORTHOTIC JOINT

(71) Applicant: Orthotic Solutions, LLC, Coon Rapids, MN (US)

(72) Inventors: Thomas Kramer, Andover, MN (US); Patrick Scott Hinshon, Maplewood, MN (US)

(21) Appl. No.: 14/814,141

(22) Filed: Jul. 30, 2015

Related U.S. Application Data
(60) Provisional application No. 62/131,347, filed on Mar. 11, 2015.
(60) Provisional application No. 62/031,152, filed on Jul. 30, 2014.

(51) Int. Cl. A61F 5/01

(52) U.S. Cl. CPC A61F 5/0127; A61F 2005/0197; A61F 2005/0151

(57) ABSTRACT

An improved AFO joint is described herein with improved stability and anti-rotational features. In particular, the AFO joint comprises a body portion with a medial beam for support and improved patient gait. Additionally, the AFO described herein comprises a tensioning member with high strength and a low profile for improved biomechanical effect.

### BACKGROUND

There are a number of pathologies that can lead to loss of function of the foot and ankle. A non-limiting list of pathologies include muscular dystrophy, multiple sclerosis, cerebral palsy and peripheral vascular disease. Often the biomechanical deficit will involve loss of the ability to bring the foot up (i.e. dorsiflexion). This situation is generically known as drop foot.

One solution is the use of an orthosis, such as the articulated Ankle Foot Orthosis (“AFO”). This orthosis has an upper portion (calf section) connected to a lower portion (foot section) by a joint. The joint is internally or externally spring loaded so that it picks up the foot and/or prevents the foot from dropping. For a spastic patient, a range limiting joint design may be indicated. A range limiting joint limits the patient's ability to push the foot down (plantarflex) beyond a predetermined angle. The articulated design allows for better biomechanical movement of the foot.

U.S. Pat. No. 5,826,304 describes a composite flexure unit for hingedly joining two relatively movable parts. The unit includes a flexure member comprising a low modulus of elasticity material. The flexure has two mounting portions and a middle connecting portion. The flexure is bendable for pivoting about a rotational axis passing through the middle portion. A load bearing element comprises a high modulus of elasticity material for providing longitudinal strength and stiffness, without significantly increasing flexion stiffness about the rotational axis. An improvement over the Carlson patent is known and marketed under the trade name Tamarak Variable Assist™ Joint, (available from Tamarack Habilitation Technologies, Inc, Blaine, Minn., USA) wherein an adjustable hinge is added to one of the mounting portions, to allow mounting the flexure unit at adjustable angles to a portion of the brace.

U.S. Pat. No. 4,665,904 to Lerman discloses a supportive brace which includes lateral and medial circular hinges rotatably securing the lateral and medial sides of the leg-supporting shell to the foot supporting shell. The circular hinges are formed by relatively large area wall portions of the shells which overlie each other in the vicinity of the ankle bones projected from the lateral and medial sides of the ankle.

U.S. Pat. No. 7,682,322 describes an articulated orthosis having at least one adjustable joint for articulating two hinged parts of the orthosis, the joint comprises a tensor for carrying the load applied between the two hinged parts. Compression surfaces coupled to the hinged parts are constructed to apply compression forces to a compression element when the angle between the two parts widens. Preferably the compression element comprises a block of resilient material. The joint allows adjustability of the unloaded angle between the hinged parts by varying the dimensions of the block, while selecting blocks having different compressional characteristics such as modulus of elasticity allows varying the degree of resistance to widening the angle between the two hinged parts.

In view of the aforementioned there is a need for an AFO joint design that allows for adjustability and is biomechanically designed for shock absorption and anti-flexion to help with knee stability upon heel contact (i.e. heel strike). Further, there is a need for an AFO with structural characteristics which resist torsional rotation of an AFO when placed in a brace. Further, there is a need to provide an AFO with an improved tensioning member that includes a low profile that limits rotation of brace components and enhances tensioning at the interface of the brace components.

### BRIEF DESCRIPTION OF THE DRAWINGS

FIG. 1 shows a top perspective view of an AFO Joint.

FIG. 2 is a top plan view of the AFO Joint as shown in FIG. 1.

FIG. 3 is a side elevation view of the AFO Joint as shown in FIG. 1.

FIG. 4 is a bottom plan view of the AFO Joint as shown in FIG. 1.

FIG. 5 is a front elevation view of the AFO Joint in FIG. 1.

FIG. 6 is a top perspective view of a tension member.

FIG. 7 is a side elevation view of the tension member of FIG. 6.

FIG. 8 is a top plan view of the tension member of FIG. 6.

FIG. 9 is a bottom plan view of the tension member of FIG. 6.

FIG. 10 is a top perspective view of a mold blank.

FIG. 11 is a top plan view of the mold blank as shown in FIG. 10.

FIG. 12 is a side elevation view of the mold blank as shown in FIG. 10.

FIG. 13 is a bottom plan view of the mold blank as shown in FIG. 10.

FIG. 14 is a front elevation view of the mold blank in FIG. 10.

FIG. 15 is a perspective and partially transparent view of another embodiment of an AFO Joint.

FIG. 16 is a top partially transparent view of the AFO Joint shown in FIG. 15.

FIG. 17 is a perspective view of a resistance member used in the alternative embodiment of FIG. 15.

FIG. 18 is a top view of the resistance member of FIG. 17.

FIG. 19 is a side view of the resistance member of FIG. 17.

### DETAILED DESCRIPTION

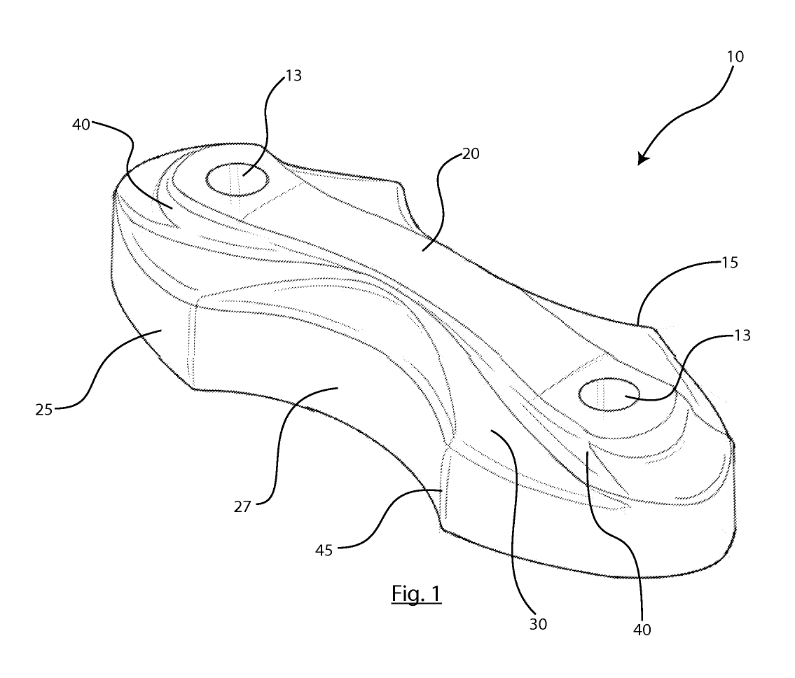

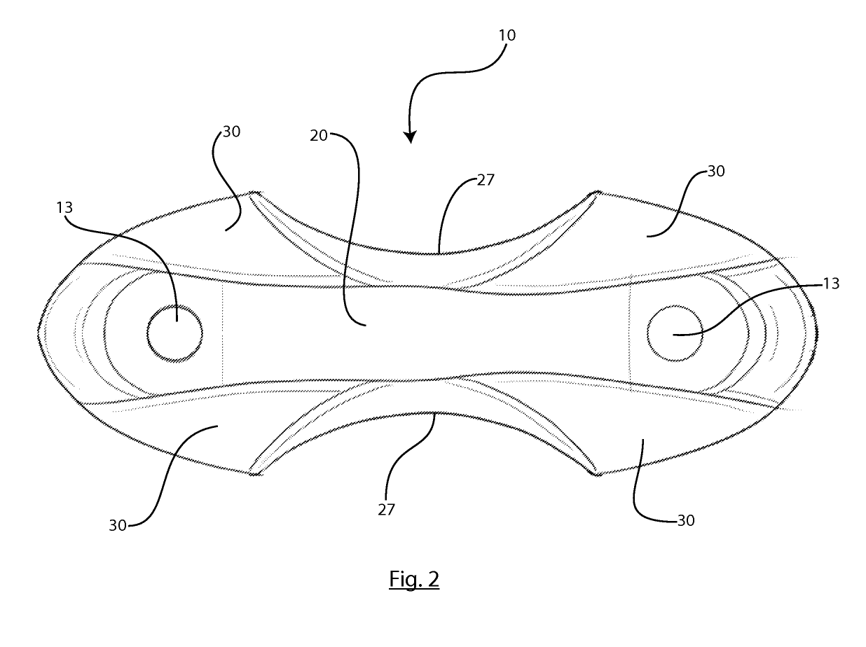

Referring now to the drawings, wherein like reference numerals designate identical or corresponding parts throughout the several views. As best illustrated in FIGS. 1-3 the disclosed embodiment of an AFO joint 10 generally includes a body 15, a tension aperture 13 and a tension member 50 (described below). As discussed in further detail below, AFO joint 10 is configured in a manner to provide a single body external structure used in conjunction with an orthotic, such as a brace. In an embodiment, AFO joint 10 can be used to provide support for and limit the range of motion of the respective leg and foot portions of an ankle foot orthotic.

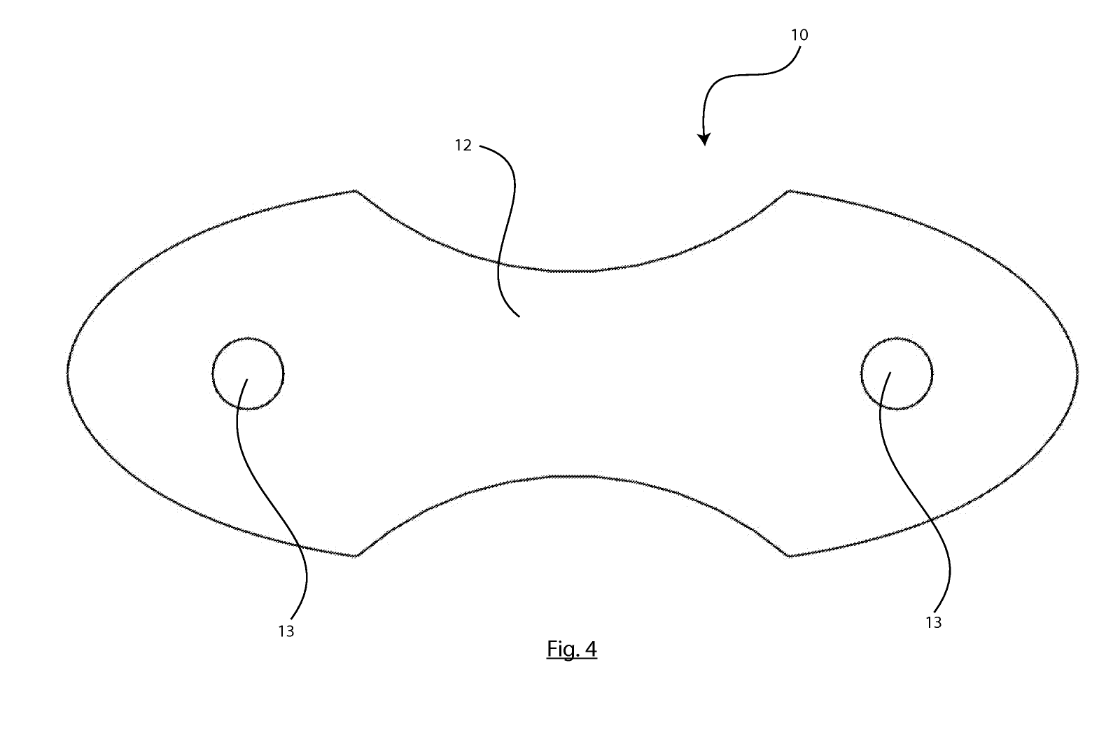

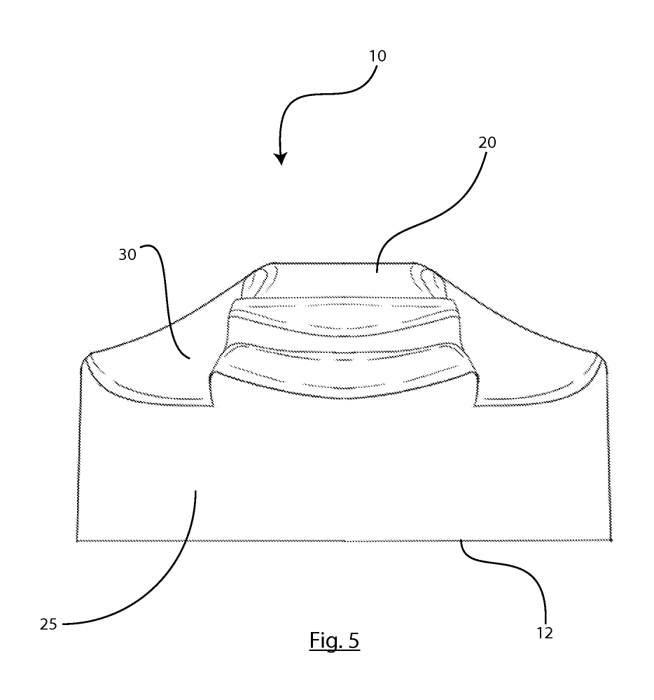

Body 15 typically comprises a beam 20, a sidewall 25, an angular wall 30 and a base 12. These several portions form a single structure for supporting a tension member 50. As shown in FIG. 1-5, body 15 may be made of silicon, polyurethane, polypropylene, and/or other similar materials. Other materials, such as composite plastics and injection molded plastics, may also be used to form body 15.

As best shown in FIG. 1, an elongate surface such as beam 20 is positioned along the medial section of body 15. Although many configurations are within the scope of the invention, beam 20 is positioned between two opposing tension apertures 13 which will support the hub 60 of tension member 50. Referring now to FIGS. 1, 3 and 5, beam 20 is shown with a curved outer surface. This feature will limit flexion of AFO joint 10 when in use. To further reduce movement, beam 20 is connected to angular wall 30 with an intersection plane 40 which is structured to reduce the rotational movement of AFO joint 10 when operably attached to a brace. The intersection point between angular wall 30 and beam 20 with intersection plane 40 may be a sharp corner or curved to further secure AFO joint 10 to a brace.

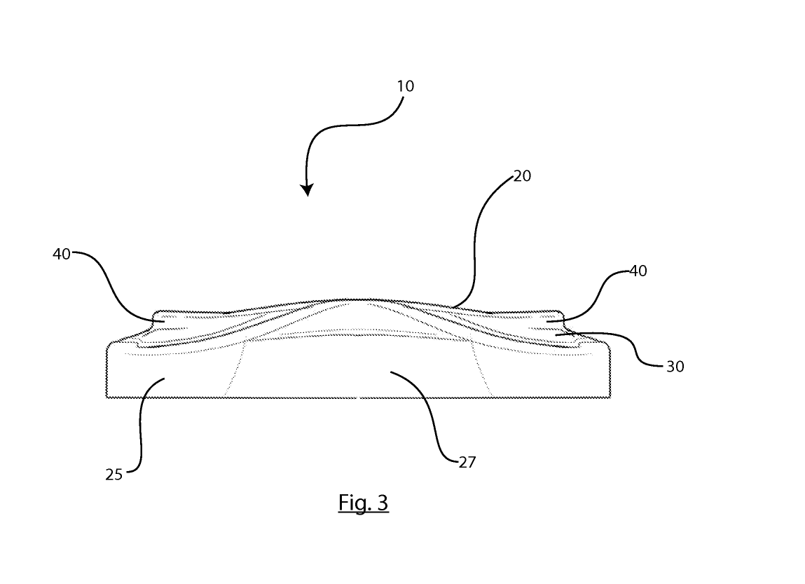

Sidewall 25 runs along the side periphery of the AFO joint 10. As best shown in FIGS. 1, 2 and 4, sidewall 25 comprises concave portions 27 which terminate along sidewall 25 to form an apex 45. Concave portions 27 permit flexion of body 15 within the transverse plane shown in FIG. 3. This structural arrangement will further improve the stability of AFO joint 10 when in use. As best shown in FIG. 1, sidewall 25 is rounded at the front and back ends in a symmetrical manner. However, it should be appreciated that the shape and configuration of the sidewall 25 can modified to provide additional support and functionalities.

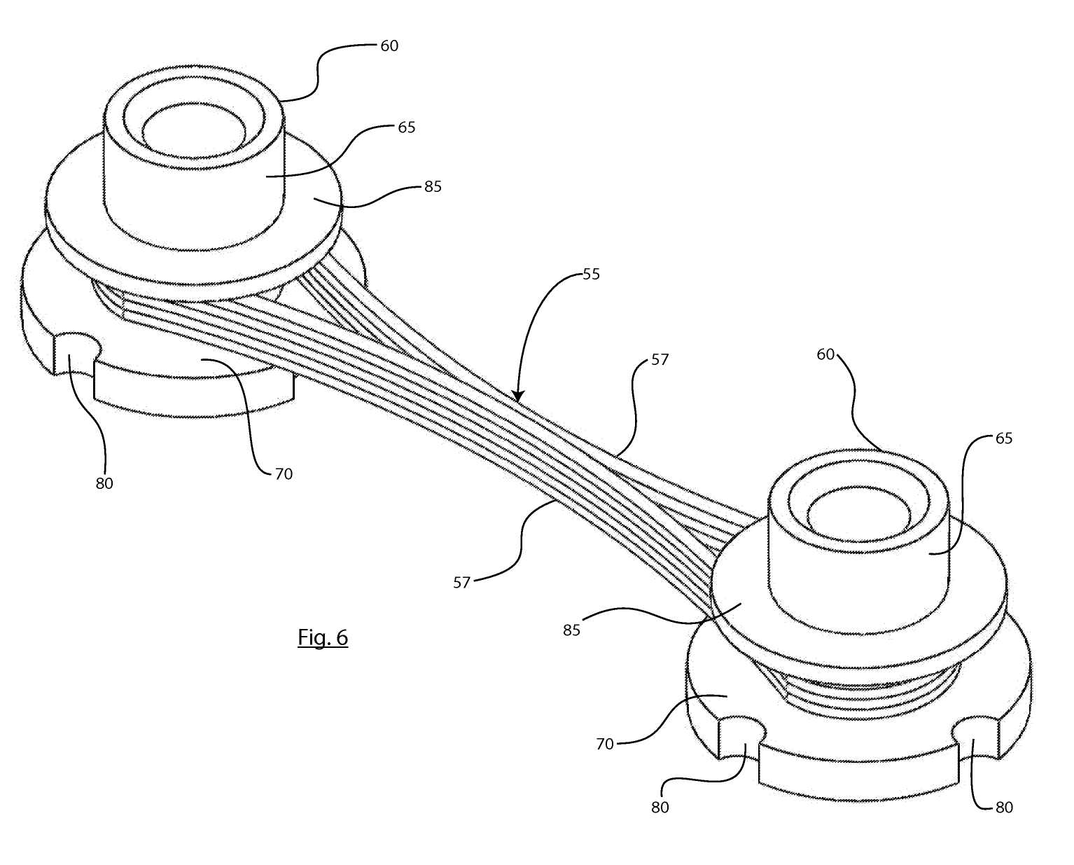

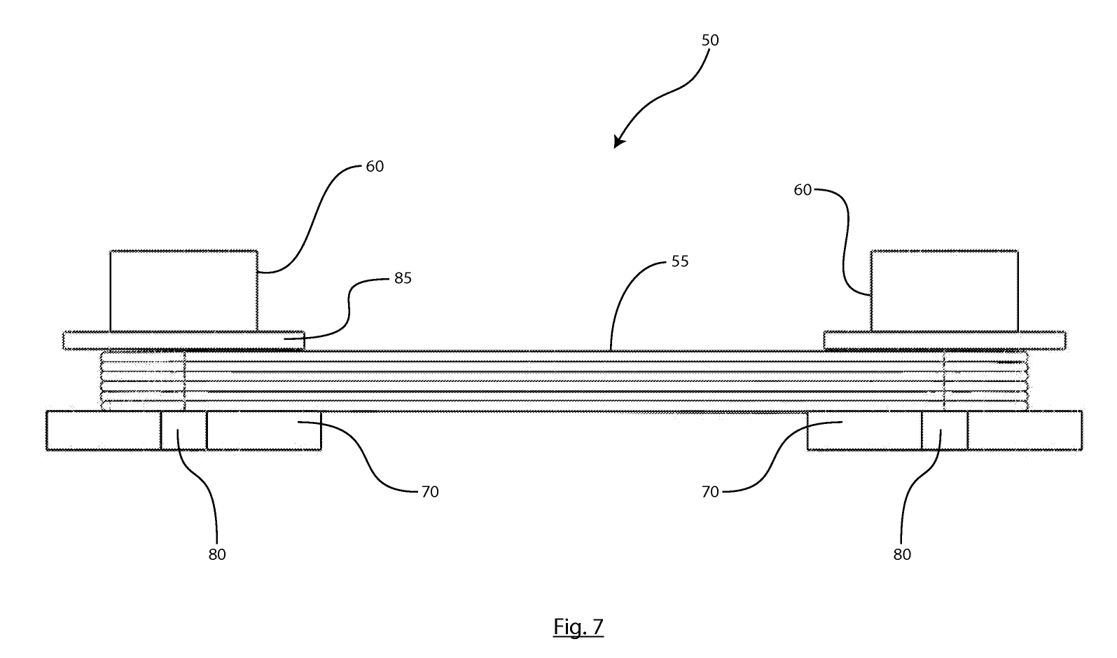

As shown in FIGS. 1, 2 and 4, tension aperture 13 is positioned on two opposed end portions of AFO joint 10. Tension aperture 13 provides an access point to tension member 50 when AFO joint 10 is being adjusted for a particular patient. Referring now to FIGS. 6-9, tension member 50 generally comprises hubs 60 and a connector, such as band 55, positioned therebetween. Tension member 50 is positioned within the internal portion of body 15 and provides tensile strength to the AFO joint 10. In an embodiment, the connector is a band 55 comprised of wrapped wire to allow for high strength and a low profile. Band 55 may be formed from wrapped wire that is shaped and hardened. Band 55 can be pinched in the generally medial portion to improve flexibility and elongation. As best shown in FIGS. 6, 8 and 9, band 55 is pinched to make an inflection region, but is not tied or fixed to a center area. The two vertical beams 57 are allowed to straighten before a static load maxes out the length of the wrapped wire. In another embodiment (not shown), band 55 is made from a metal that will bias the AFO joint back to a straight position every time that the AFO joint is flexed.

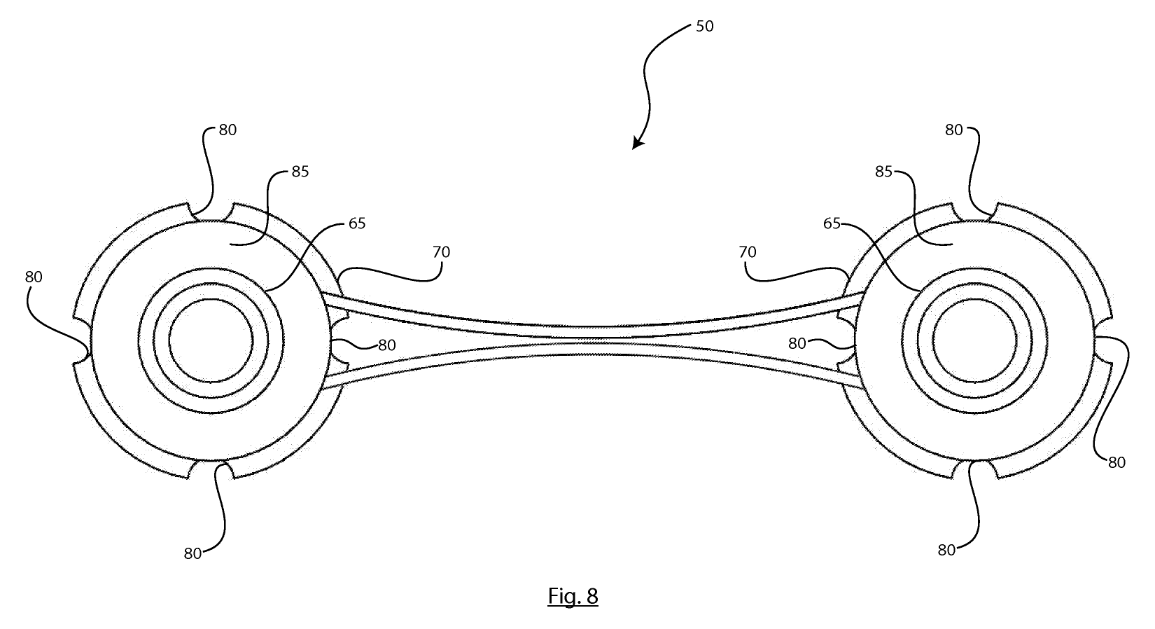

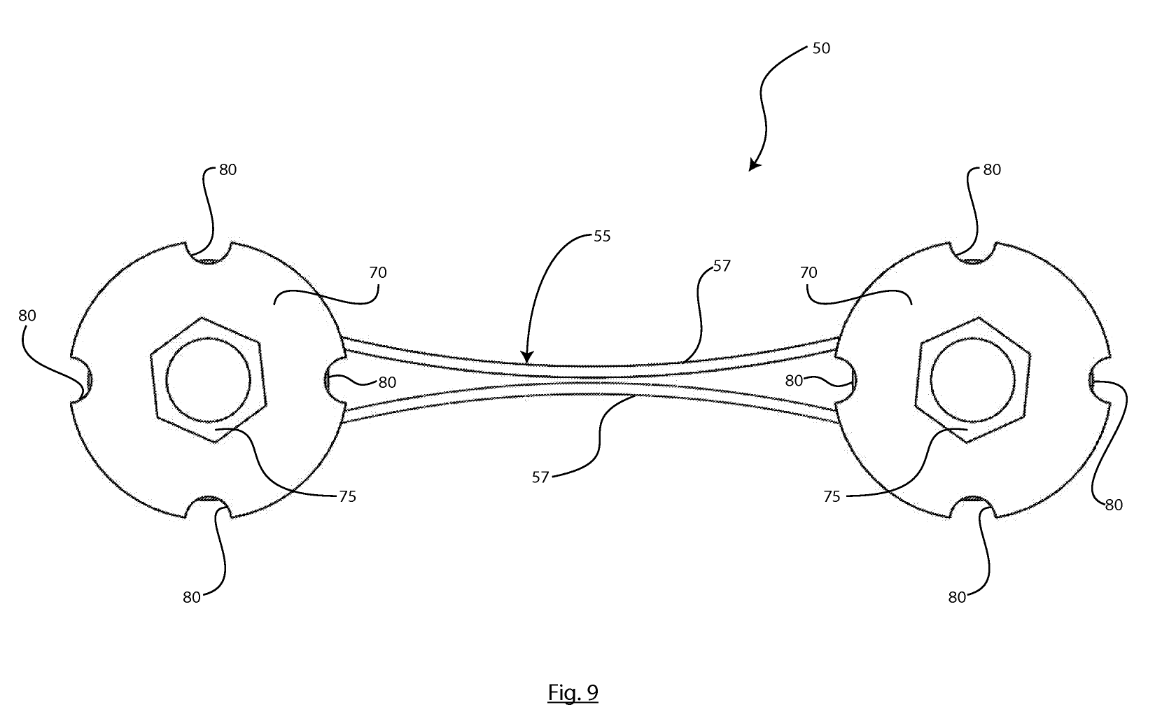

Hub 60 generally comprises a neck 65, a base plate 70 and a support plate 85. Neck 65 generally has an elongated columnar shape and defines a threaded cavity adapted to receive a complementary threaded member. The elongated neck 65 provides an enhanced threading interface for adjusting the AFO joint 10. Base plate 70 and support plate 85 retain band 55 and prevent the sliding of band 55 along neck 65. Referring now to FIGS. 6 and 9, base plate 70 generally includes at least one side recess 80. Side recess 80 is generally adapted to engage complementary structure integrated into a portion of the brace, such as a boss located on an outer surface of the brace. In an embodiment, the boss occupies recess 80, thereby interfering with and substantially inhibits further rotation of hub 60. Although no particular number of side recesses 50 is preferred, the embodiment shown in FIGS. 8 and 9 depict base plate 70 with four side recesses 80. Referring specifically to FIG. 9, base plate 70 may further comprise a hex insert 75 to allow tension member 50 to receive and be supported by an Allan wrench or similar tool while forming the AFO joint 10.

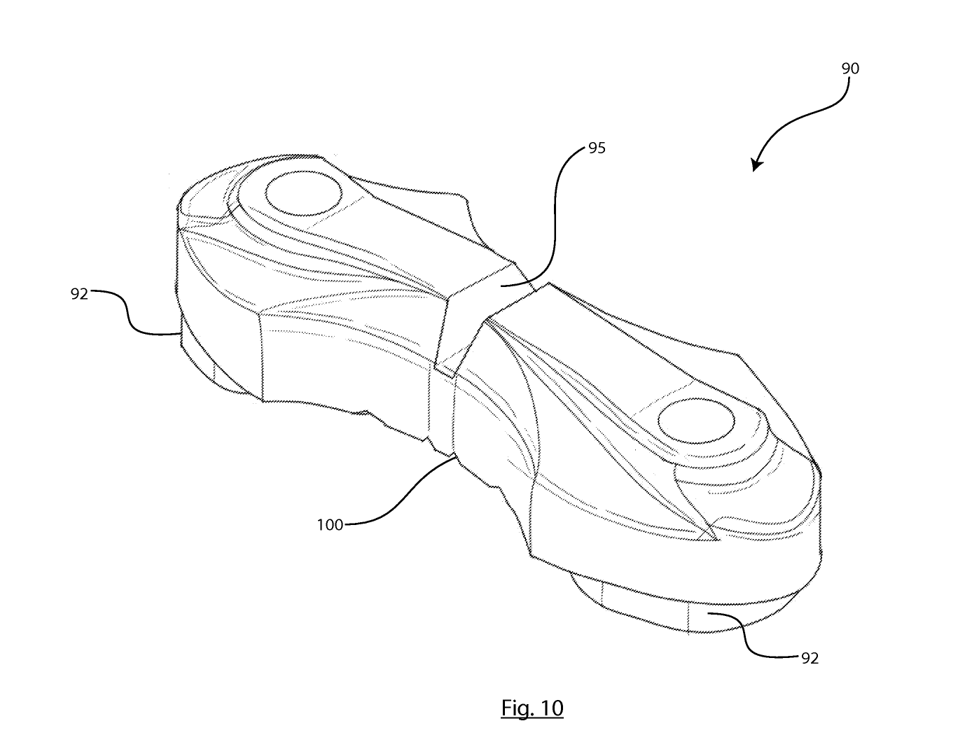

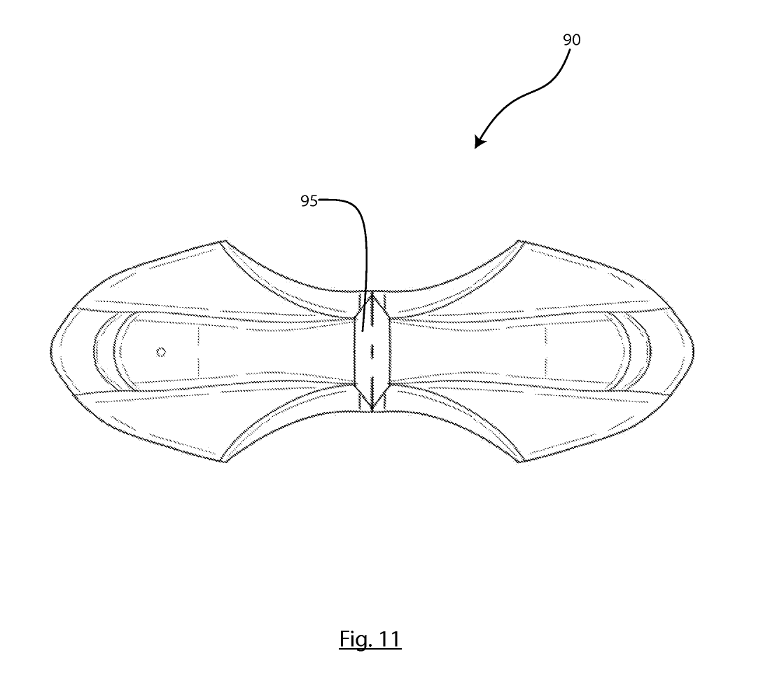

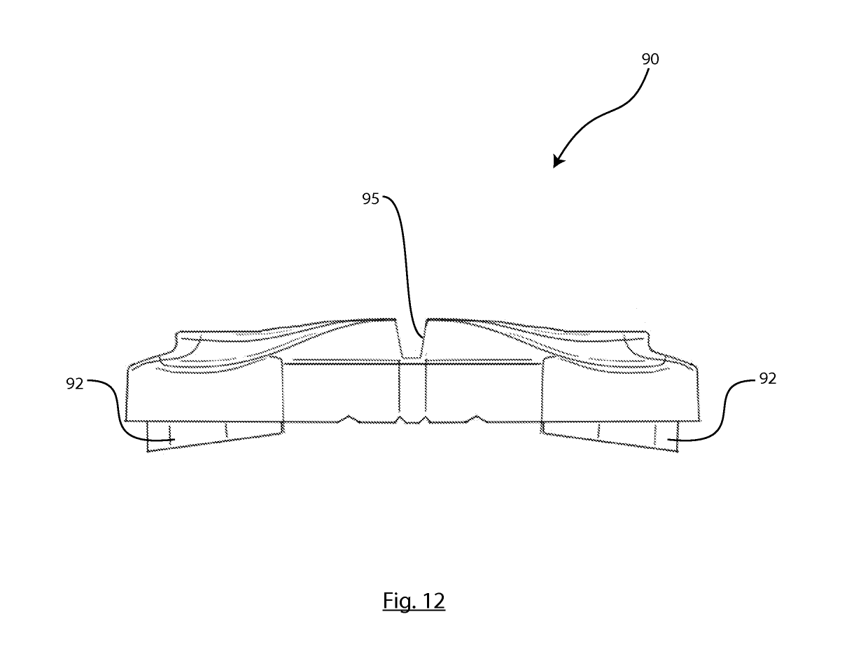

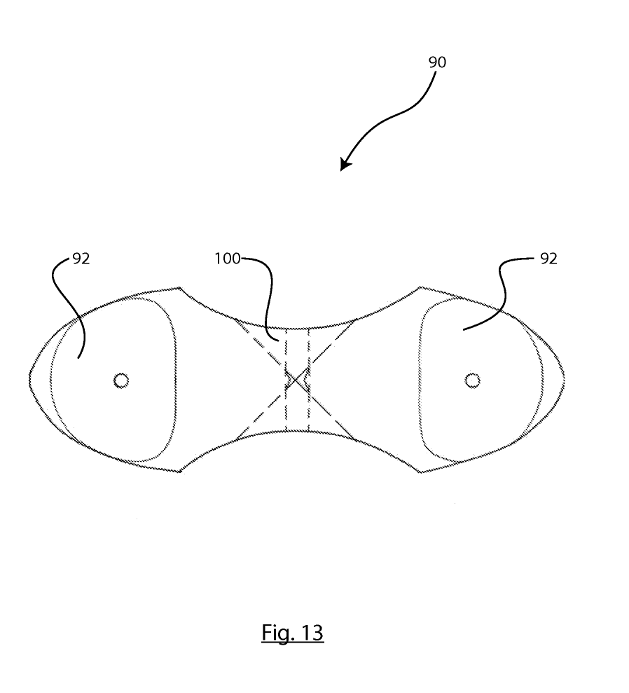

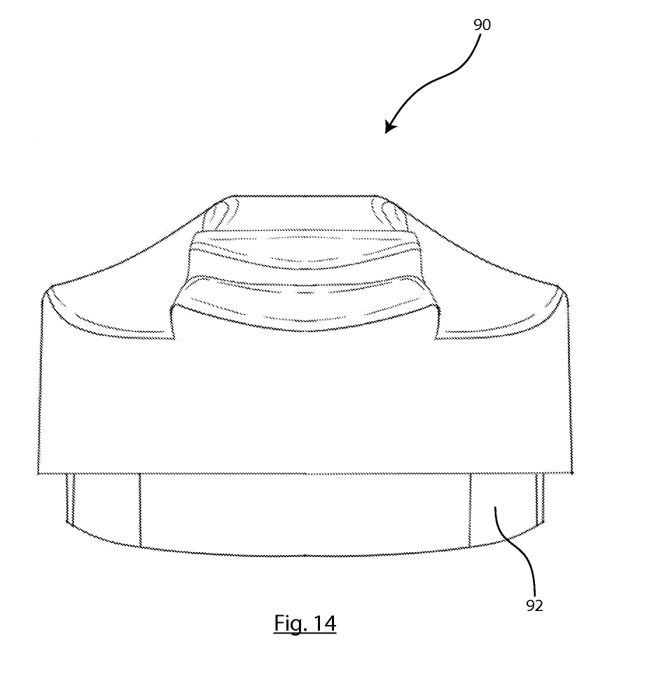

Referring now to FIGS. 10-14, which depict AFO joint mold blank 90 similar to AFO joint 10 described above. When AFO is being formed, AFO joint mold blank 90 is positioned with respect to an AFO to create a void that will ultimately be occupied by AFO joint 10. Although several of the portions of the AFO joint mold blank 90 are similar to AFO joint 10 in structure, the AFO joint mold blank 90 provides limited functional benefit to an AFO. To state another way, AFO joint mold blank 90 does not include a tension member 50 or like structure, but only similar surface contours to help provide a relatively precise void for the AFO joint 10. Referring now to FIGS. 10, 13 and 14, AFO joint mold blank 90 comprises foot pads 92 which will allow for a more comfortable contour along a patient's anatomy when the AFO joint 10 is positioned along the ankle joint.

AFO joint mold blank 90 also includes a channel 95 which can be used to facilitate breaking and removing the AFO joint mold blank 90 once the AFO is form fitted. To further expedite the removal of the AFO joint mold blank 90, the brace may include surface etching 100 which will help cleanly break the AFO joint mold blank 90 without interfering with the surrounding surfaces of the brace. These features which provide an easier removal of the AFO joint mold blank 90 and will also lessen the chance that the brace will become damaged.

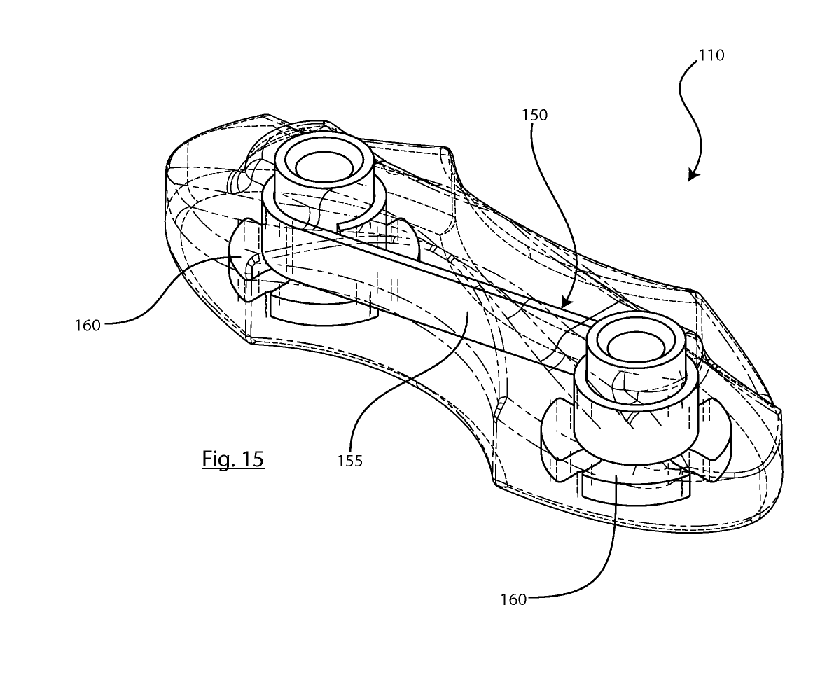

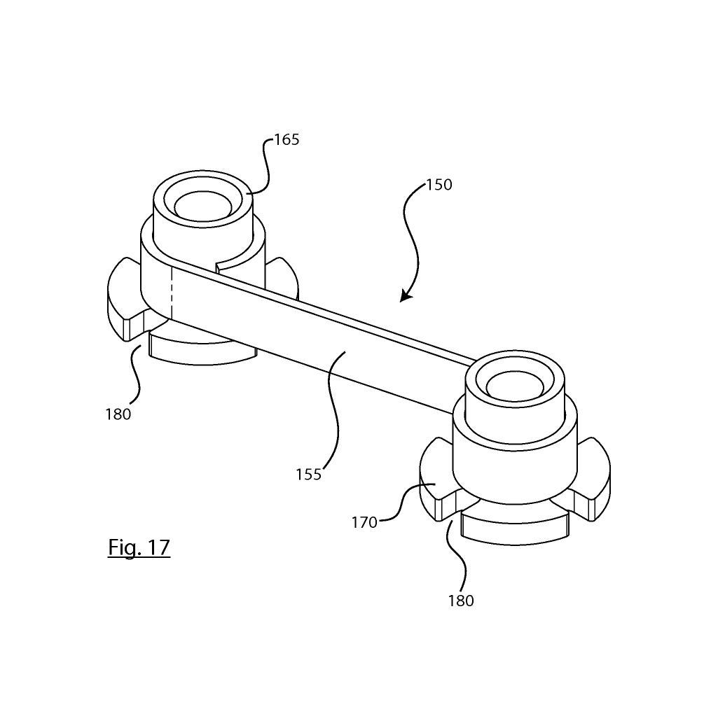

As best illustrated in FIGS. 15-19, an alternative resistance component 150 has been included in an alternative embodiment of an AFO joint 110. In this alternative embodiment, resistance component 150 includes a resistance member 155, a pair of hubs 160, and a related holding body. As best illustrated in FIGS. 17-19, resistance member 155 is a singular strip of material, which has a relatively straight central portion, and two end portions which are curved and wrapped around hubs 160. In a manner similar to the embodiment outlined above, hubs 160 include a central neck portion 165 and a base plate 170. As seen, base plate 170 also includes a number of notches 180 which are provided to assist in the positioning and holding of AFO joint 110 when in use.

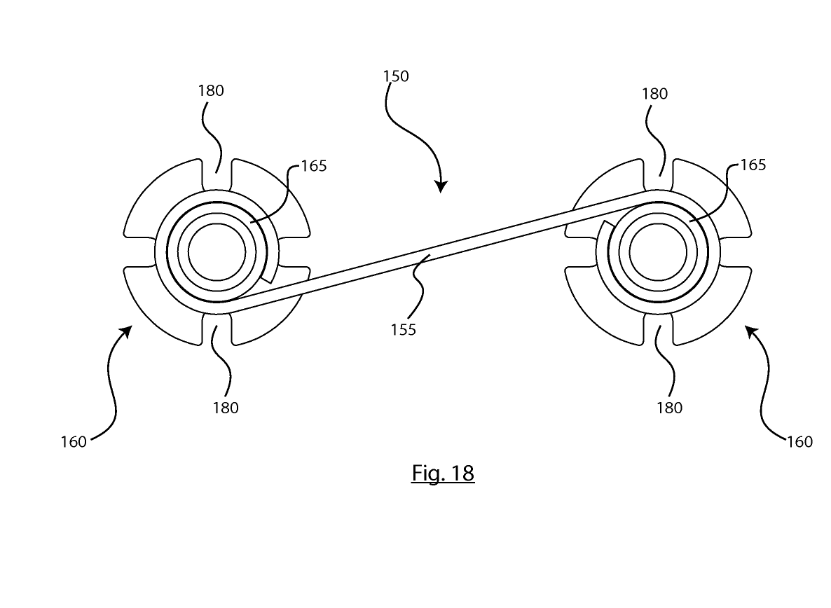

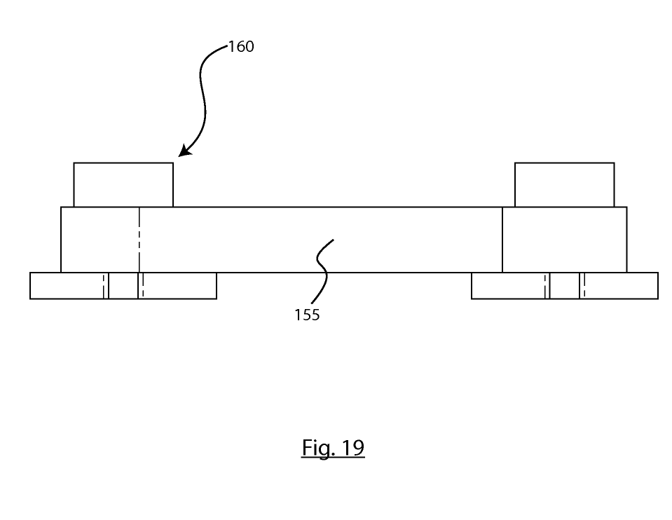

In this alternative embodiment, it is anticipated that resistance member 155 will be fabricated from a substantially rigid metal material. For example, this metal material may be the well understood and well known spring steel, which is typically used in many spring type operations or components. As best illustrated in FIG. 19, resistance member 155 has a length which is substantially larger than its width. This creates a structure which is substantially resistant to flexing or bending along one direction while capable of flexing in a perpendicular direction. The relatively thinner width is better illustrated in FIG. 18. From this top view, it is clear that resistance member 155 is narrow (relatively speaking) from a top side. Based upon this configuration, the flexing and bending of resistance member 155 and AFO joint 110 is easily controlled and well understood by those skilled in the art.

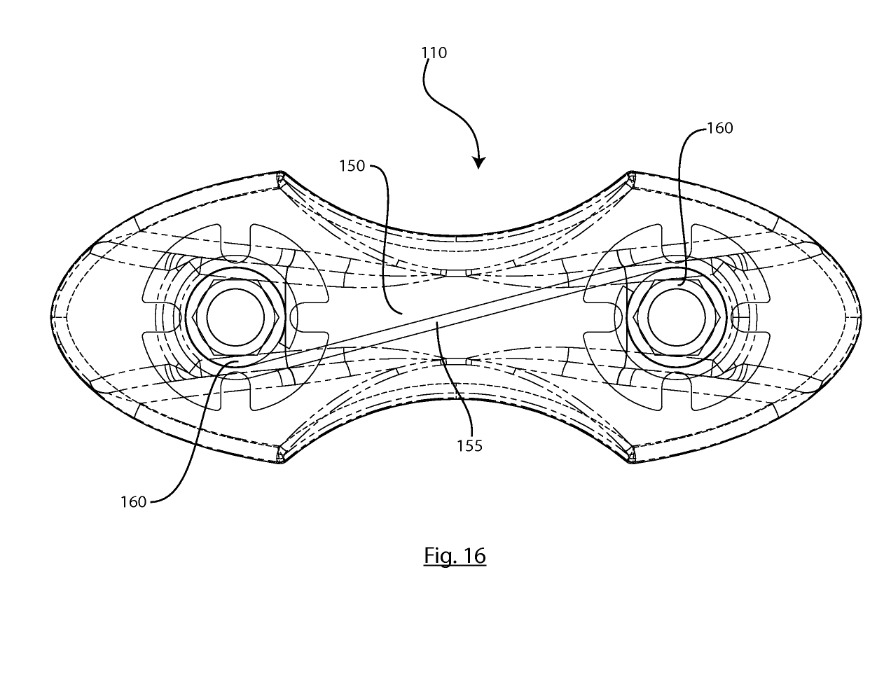

To provide context, FIGS. 15-16 illustrate this alternative resistance component 150 within a joint body 115. More specifically, joint body 115 is illustrated with dashed lines, so that alternative resistance component 150 can be better seen. It is contemplated that joint body 115 is shaped and sized in a substantially similar manner to body 15 discussed above and illustrated in FIG. 1. One skilled in the art will recognize that several alternatives are possible.

In addition, it is contemplated that the AFO joint 110 of the alternative embodiment discussed in relation to FIGS. 15-19 could be attached and adjusted in the same manner as the previous embodiments. To achieve this general commonality between embodiments, the hubs 160 and baseplate 170 are substantially the same as the hubs 60 and baseplate 70 discussed in relation to FIGS. 6-9. That said, the dimensions and proportions will be configured to closely cooperate with resistance component 150.

Various embodiments of the invention have been described above for purposes of illustrating the details thereof and to enable one of ordinary skill in the art to make and use the invention. The details and features of the disclosed embodiment[s] are not intended to be limiting, as many variations and modifications will be readily apparent to those of skill in the art. Accordingly, the scope of the present disclosure is intended to be interpreted broadly and to include all variations and modifications coming within the scope and spirit of the appended claims and their legal equivalents.

1.-15. (canceled)

16. A joint for selective attachment to an ankle foot orthosis comprising:
a body defining an inner cavity, the body having a first end, a second end, an outer wall, and a sidewall, the sidewall defining a periphery of the body;
a first tension anchor disposed substantially within the inner cavity and attachable to the ankle foot orthosis;
a second tension anchor disposed substantially within the inner cavity and attachable to the ankle foot orthosis, the second tension anchor spaced apart from the first tension anchor; and
a connector operably coupled to the first tension anchor and the second tension anchor and providing tensile strength to the joint.

17. The joint of claim 16, wherein the outer wall is substantially flat or convex.

18. The joint of claim 17, wherein the sidewall includes a first concave portion opposite a second concave portion, the first and second concave portions disposed intermediate the first and second ends such that the body is deformable along a transverse plane defined by the body when a force is applied transversely to the first end or the second end.

19. The joint of claim 18, wherein the outer wall includes an elongate surface disposed between a first angled surface and a second angled surface, the elongate surface opposing deformation of the body in response to the transversely applied force.

20. The joint of claim 19, wherein the connector establishes a limited range of deformation of the body within the transverse plane.

21. The joint of claim 20, wherein disposition of the elongate surface with respect to the first and second angled surfaces opposes torsion of the first end of the body with respect to the second end of the body.

22. The joint of claim 21, wherein the body defines a first aperture and the first tension anchor defines a first tension anchor lumen substantially aligned with the first aperture.

23. The joint of claim 22, wherein the first aperture and the first tension anchor lumen are adapted to receive an implement for rotating the first tension anchor.

24. The joint of claim 23, wherein rotating the first tension anchor in a first direction increases tension on the connector and rotating the first tension anchor a second direction decreases tension on the connector.

25. The joint of claim 24, wherein the body defines a second aperture and the second tension anchor defines a second tension anchor lumen substantially aligned with the second aperture, the second aperture and the second tension anchor lumen being adapted to receive the implement.

26. The joint of claim 25, wherein rotating the second tension anchor in the first direction or the second direction increases tension on the connector and rotating the second tension anchor in the other of the first direction or the second direction decreases tension on the connector.

27. An orthotic device for supporting a user's foot, the orthotic device comprising:
a first support portion of the orthotic device adapted to support the user's foot;
a second support portion of the orthotic device adapted to support a portion of one of the user's lower legs;
a joint operably coupled to the first portion and the second portion of the orthotic device, the joint comprising:
a body defining an inner cavity, the body having a first end, a second end, an outer wall, and a sidewall, the sidewall defining a periphery of the body;
a first tension anchor disposed substantially within the inner cavity and attachable to the ankle foot orthosis;
a second tension anchor disposed substantially within the inner cavity and attachable to the ankle foot orthosis, the second tension anchor spaced apart from the first tension anchor; and
a connector operably coupled to the first tension anchor and the second tension anchor and providing tensile strength to the joint.

28. The orthotic device of claim 27, wherein the outer wall is substantially flat or convex.

29. The orthotic device of claim 28, wherein:
the outer wall includes an elongate surface disposed between a first angled surface and a second angled surface to oppose torsion of the first end with respect to the second end, the elongate surface applying a biasing force that resists deformation of the body along a transverse plane defined by the body;
the sidewall includes a first concave portion opposite a second concave portion, the first and second concave portions disposed intermediate the first and second ends such that the body is transversely deformable along the transverse plane when a load is transversely applied to the first end or the second end; and
the connector establishes a limited range of deformation of the body within the transverse plane.

30. The orthotic device of claim 29, wherein the body defines a first aperture and the first tension anchor defines a first tension anchor lumen substantially aligned with the first aperture, the first aperture and the first tension anchor lumen being adapted to receive an implement for rotating the first tension anchor.

31. The orthotic device of claim 30, wherein rotating the first tension anchor in a first direction increases tension on the connector and rotating the first tension anchor a second direction decreases tension on the connector.

32. The orthotic device of claim 31, wherein rotating the second tension anchor in the first direction or the second direction increases tension on the connector and rotating the second tension anchor in the other of the first direction or the second direction decreases tension on the connector.

33. The orthotic device of claim 32, further comprising a second joint operably coupled to the first portion and the second portion of the orthotic device.

34. A method of manufacturing an orthotic device comprising:
providing a first joint, the first joint having an outer structure;
providing a joint mold blank, the join mold blank having an outer structure substantially similar to the outer structure of the first joint and a channel aligned transverse to first and second ends of the join mold blank;
providing first and second support portions of the orthotic device attached to the joint mold blank;
inducing a fissure along the channel to separate the first end of the joint mold blank from the second end of the joint mold blank;
removing the first and second ends of the joint mold blank from the first and second support portions of the orthotic device; and
attaching the joint to the first and second portions of the orthotic device.

35. The method of claim 34, further comprising:
providing a second joint having an outer structure;
providing a second joint mold blank, the second joint mold blank having an outer structure substantially similar to the outer structure of the second joint and a channel aligned transverse to first and second ends of the second join mold blank, the first and second portions of the orthotic device attached to the second joint mold blank;
inducing a fissure along the channel of the second joint mold blank to separate the first end of the second joint mold blank from the second end of the second joint mold blank;
removing the first and second ends of the second joint mold blank from the first and second support portions of the orthotic device; and
attaching the second joint to the first and second support portions of the orthotic device opposite the first joint.
